(19) United States
(12) Patent Application Publication
(10) Pub. No.: US 2009/0104586 A1
(43) Pub. Date: Apr. 23, 2009

(54) Collagen Antral Membrane Expander

(75) Inventors: Ronald F. Cardoso, Sao Paulo (BR); Dennis Smiler, Encino, CA (US); William K. Knox, Newport Beach, CA (US)

Correspondence Address: CROWELL & MORING LLP;INTELLECTUAL PROPERTY GROUP, P.O. BOX 14300, WASHINGTON, DC, 20044-4300, US

(73) Assignee: Osseous Technologies of America, Newport Beach, CA (US)

(21) Appl. No.: 12/256,995

(22) Filed: Oct. 23, 2008

Related U.S. Application Data
(62) Division of application No. 11/444,356, filed on Jun. 1, 2006.
(60) Provisional application No. 60/686,052, filed on Jun. 1, 2005.

(51) Int. Cl. A61B 5/117; A61M 29/00

(52) U.S. Cl. 433/229; 606/192

(57) ABSTRACT

A subantral membrane elevator comprising an inflatable balloon and an over-formed membrane (e.g., collagen membrane), which is resorbable into the body, and a method of using the same in bone augmentation surgery, such as alveolar bone augmentation preparatory to implantation of a dental implant.

### CROSS-REFERENCE TO RELATED APPLICATIONS

This application is a divisional application from U.S. application Ser. No. 11/444,356, filed Jun. 1, 2006, which claims the priority of non-provisional application no. 60/686,052, filed Jun. 1, 2005, the disclosures of which are expressly incorporated by reference herein.

### BACKGROUND OF THE INVENTION

1. Field of the Invention

The present invention relates generally to an apparatus and method for dental surgery, and particularly to subantral augmentation.

2. Description of Related Art

Osseointegrated fixtures are commonly used to treat edentulous patients. However, a concern when restoring the edentulous posterior maxilla is an atrophic posterior maxillary alveolus combined with a large maxillary sinus. An edentulous posterior maxilla with alveolar resorption together with increased pneumatization of the sinus can limit the quantity and quality of bone available for successful implant placement. Specifically, crestal alveolar bone loss combined with increased pneumatization of the sinus diminishes the amount of alveolar bone available for satisfactory placement of osseointegrated implants and oral reconstruction.

When bone is inadequate for implant placement, particular concerns for clinician and patient are risk of lost implant anchorage, failure of implants under function, penetration into the sinus, development of an oral-antral opening and/or infection. Successful incorporation of implants generally requires about 10 mm or more of alveolar bone height, while radiographs of patients targeted for subantral augmentation often reveal a bone partition between crestal bone and sinus of as little as two to three millimeters. While shorter implants can be placed in the atrophic posterior maxilla, this often results in an unfavorable crown to root ratio of the restored implant supported prosthesis. This situation combined with increased destructive occlusal forces may adversely influence the long-term success of a patient's implant-supported restoration.

Lifting the sinus membrane and performing subantral bone graft augmentation on the floor of the sinus provides an osteoconductive and osteophilic structure having sufficient quantity and quality of bone for the placement of osseointegrated implants and oral rehabilitation. However, the success of the bone graft depends to a large extent on maintaining the integrity of the sinus membrane.

The sinus lift subantral augmentation procedure is an accepted technique to treat the loss of vertical bone height in the posterior maxilla. However, when the edentulous area is between one or two teeth, lifting the membrane can be difficult and the membrane is more easily torn.

Accordingly, it would be advantageous to provide a procedure for subantral augmentation that facilitates ease of access to the edentulous region and minimizes the likelihood of tearing the sinus membrane.

### SUMMARY OF THE INVENTION

It is an object of the invention to provide an improved subantral augmentation apparatus and procedure.

It is a further object of the invention to provide an improved antral membrane expander, which will minimize the likelihood of tearing the sinus membrane.

Another object of the invention is to provide an improved method of augmenting bone, particularly alveolar bone.

In accordance with a first aspect of the invention, these objects are achieved by providing an antral membrane expander comprising an inflatable balloon having a single opening fitted with a connector for an inflating means. A membrane of resorbable material is fitted over the balloon, and the balloon is used to incorporate the resorbable material within a bone graft site.

In another aspect of the invention, the objects are achieved by providing a method of augmenting bone in a living organism comprising forming an incision to access a bone graft site between a bone to be augmented and an overlying membrane, inserting an inflatable balloon into the bone graft site, inflating the balloon to form a cavity between the bone and the membrane, deflating and removing the balloon from the cavity, fitting a resorbable membrane over the deflated balloon to form a membrane-encapsulated balloon, inserting the membrane-encapsulated balloon into the cavity and re-inflating the balloon to press the resorbable membrane material against an inner wall of the cavity to form a resorbable membrane-modified cavity, deflating and removing the balloon, filling the membrane-modified cavity with a bone augmentation material, and closing the incision.

### BRIEF DESCRIPTION OF THE DRAWINGS

The invention will be describe in further detail hereinafter with reference to illustrative preferred embodiments shown in the accompanying drawing figures in which:

FIG. 1 is a perspective view of an antral membrane expander according to the invention in the un-inflated state;

FIG. 2 is a perspective view of the antral membrane expander of FIG. 1 in an inflated state;

FIG. 3 is a perspective view of an antral membrane expander according to the invention inserted through an implant receptor site; and

FIG. 4 is a perspective view of an antral membrane expander according to the invention inserted through a buccal window.

FIGS. 5-10 illustrate a sinus lift procedure using a collagen antral membrane expander.

### DETAILED DESCRIPTION

The collagen antral membrane expander technique elevates the schneiderian membrane and accesses the subantral space for graft augmentation. An inflatable balloon is used to elevate the membrane. The inward reflection of the osteotomy, dissection of the membrane from the antral floor, and inflation of the balloon result in an antral space bordered superiorly by the reflected buccal bone and membrane, medially by the medial wall of the sinus, and anterior/posterior by the non-reflected membrane and the roots of the adjacent teeth.

After creation of the subantral space, but before insertion of the graft material therein, a resorbable membrane is inserted into the subantral space using the inflatable balloon and is incorporated onto an exposed surface within the cavity. Specifically, the inflatable balloon is deflated, encapsulated with the resorbable membrane, inserted into the cavity, and inflated to press the resorbable membrane material against an inner wall of the cavity. The resorbable membrane remains within the cavity upon removal of the balloon.

This sinus lift technique, which elevates the sinus membrane easily and accesses the subantral space for graft augmentation, is advantageously accomplished with limited incision and mucoperiosteal flap reflection. The sinus membrane can be elevated to the medial wall of the sinus without sharp dissection around the roots of adjacent teeth. Further, with this procedure there is reduced morbidity, reduced blood loss, reduced operative time, and reduced post operative pain and complications. The technique is especially useful when the edentulous area is surgically difficult to access such as between one or two teeth.

The surgery can be performed from the quadrilateral window, a small buccal osteotomy, or a crestal approach through an implant receptor site. The balloon is preferably positioned midway between the buccal bone and the medial wall of the sinus and slowly inflated with 1 to 6 cc of sterile water or physiological saline solution. As the balloon expands, the membrane is elevated upward from the antral floor. Care is taken to avoid tearing or rupturing the sinus membrane.

The success of the operation and bone graft depends on maintaining the integrity of the antral membrane. The membrane lining the sinus is a mucous membrane of the respiratory type containing pseudostratified, columnar, ciliated epithelium. The mucociliary mechanism can be impaired, paralyzed or permanently damaged by trauma or tearing from surgical perforation. When the lining mucous membrane is torn, blood collects on the sinus floor dispersing the graft material. If infection ensues there may be a foul discharge and pus into the graft and oral cavity. Further, if the integrity of the membrane is breached there is the risk that the graft material will not be contained and will dissipate through the open membrane into the sinus cavity and possibly into the oral compartment. Advantageously, the collagen antral membrane expander technique acts to patch any tear within the sinus membrane. This reduces and/or eliminates the risk of infection, dispersal of graft material (i.e., into the sinus cavity) and failure of the graft to integrate.

The resorbable membrane material is preferably made of an engineered collagen-based matrix. Collagen (e.g., type I collagen) can be derived (isolated and purified) from either a bovine (e.g., bovine corium), porcine, equine, or synthetic source. Collagen can also be derived from Achilles tendon. The extraction process involves a sequential extraction of non-collagenous material, and the sequential removal of non-collagenous moieties from the tissue to produce an insoluble, intact collagen. The intact collagen fibers retain their intrinsic intermolecular crosslinks and the fibrils are arranged in their native configuration. The resorbable membrane is manufactured as a nonfriable and conformable membrane matrix that can be fitted over the inflatable balloon. The engineered collagen-based matrix forms a fibrous network on which new cells can grow.

In addition to the above-identified sources, collagen useful to make the resorbable membrane can also be obtained by recombinant methods. Recombinantly-produced type I collagen is commercially available, for example, from FibroGen, Inc., of South San Francisco, Calif.

Further advantages of using insoluble collagen fibers (e.g., type I collagen) for the resorbable membrane include:
Human collagen is homologous to animal collagen and therefore well accepted for implantation.
Collagen-based implants have been used for tissue and organ repair and regeneration for the past two decades without adverse effects.
Collagen fibers can be engineered into a variety of matrix types.
Collagen-based products can be cost effectively manufactured in large quantities.
Collagen is available in various shapes and sizes.
Collagen is involved in development and wound healing processes in the body.
Collagen is currently used as a hemostatic wound dressing.
Resorbable collagen is currently used for guided tissue and bone regeneration in oral surgery to assist in wound healing.
Cells and bioactive agents (growth factors, platelet rich plasma) can be incorporated into collagen for enhanced healing.
Collagen is resorbable (broken down by the enzyme collagenase). The breakdown products are short peptides and amino acids that are eliminated via normal metabolic pathways.
Collagen can be programmed for resorption from 2-3 weeks, 6-9 months or years depending on the needs of the particular membrane application.

The method of the invention is a surgical procedure for repairing and/or preventing tears of the sinus membrane immediately prior to placing the graft material. This is accomplished with an antral membrane expander comprising an inflatable balloon which, after forming the subantral cavity, is deflated and removed from the cavity, encapsulated in a resorbable collagen-based membrane material, re-inserted into the cavity, and re-inflated in order to incorporate the resorbable membrane material on an exposed inner surface of the cavity.

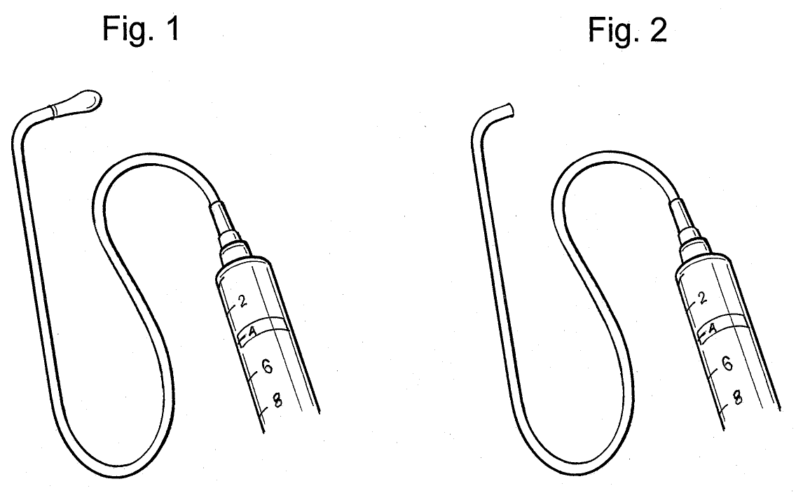

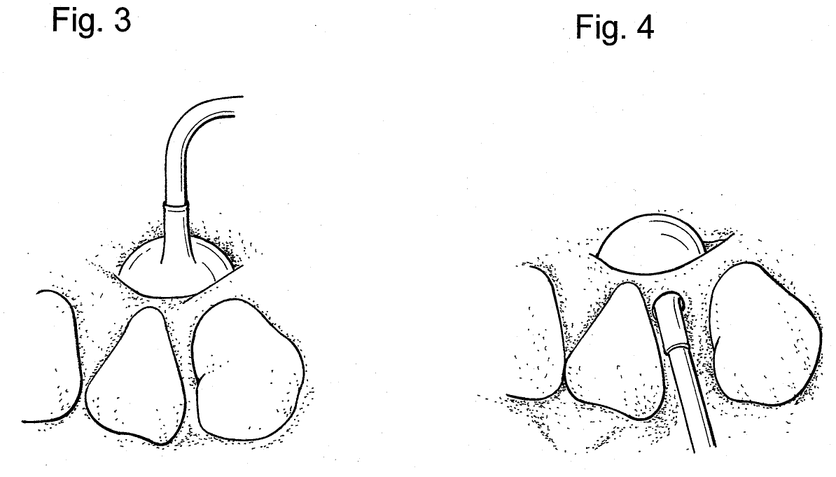

The antral membrane expander comprises an inflatable balloon that is attached to a suitable connector such as a luer-lock type connector. The balloon is attached via the connector and a length of tubing to an inflating means such as a liquid-filled syringe. FIG. 1 shows an antral membrane expander where the balloon is in an unexpanded state. FIG. 2 shows an antral membrane expander where the balloon is in an expanded state.

The balloon may be provided in various sizes depending on the desired volume of bone augmentation. For example, the user may selected a device with an initial volume of from about 1 cubic centimeter to about 6 cubic centimeters (e.g., 1, 2, 3, 4, 5 or 6 cc) depending on the proposed surgery graft site.

In use, local anesthesia is obtained with infiltration of the buccal and palatal tissues. A crestal incision is made extending the length of the edentulous area. If the attached keratinized gingiva is narrow, the incision is made slightly palatal to the crest. At the anterior border of the crestal incision, a vertical wide-based relaxing incision is angled forward and extends to the vestibule.

A full thickness mucoperiosteal flap is dissected and reflected superiorly exposing the buccal bone beyond the muco-gingival line. The flap is elevated around the existing teeth to expose the lateral wall of the maxilla. The position and outline of the maxillary sinus can be determined using a panographic x-ray. If the buccal wall is thick, transillumination of the sinus from the palatal side can help visualize the location of the sinus floor and the anterior border.

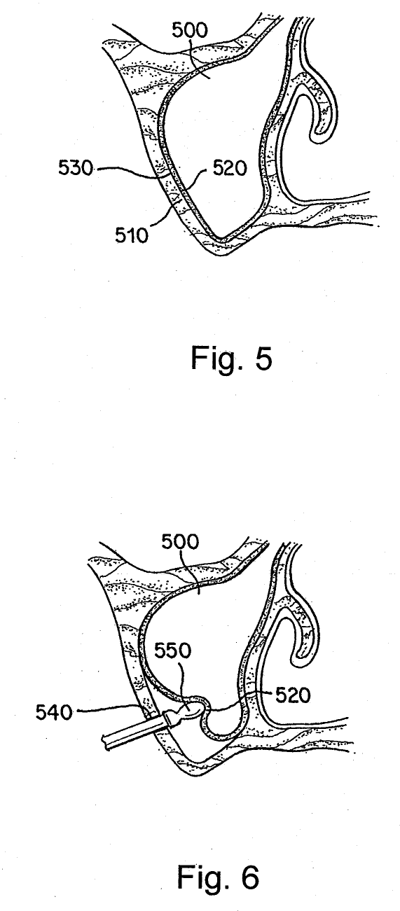

An osteotomy of the buccal bone is performed under copious irrigation with a trephine or a #6 round bur. Preferably, the osteotomy is carried through the cortical bone without tearing the sinus membrane. The oval osteotomy with the sinus membrane is gently pressed inward or is removed exposing the intact membrane. A large curette or modified sharp Freer elevator can be used to dissect and separate the membrane from the antral floor. The dissection progresses to the medial wall of the sinus. An inflated balloon 300 inserted via a buccal window 310 is shown in FIG. 3.

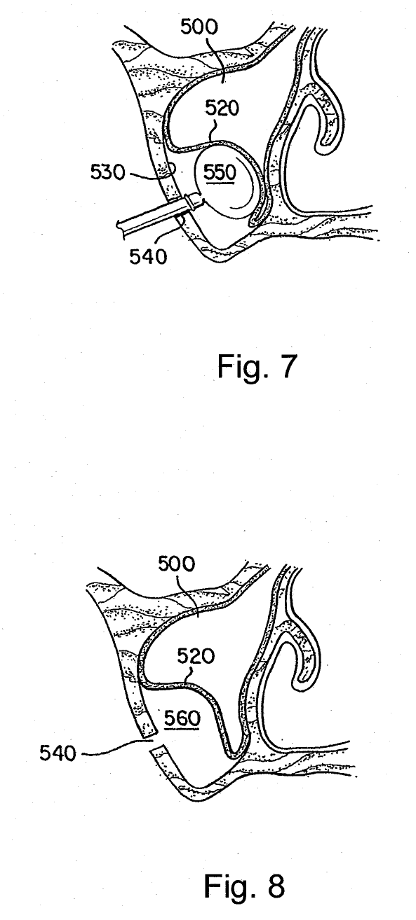

As illustrated in FIG. 4, an alternative procedure is accomplished from a prepared implant receptor site of the crestal bone. FIG. 4 shows inflated balloon 400 inserted via implant receptor site 410. In this procedure, an incision is made on the palatal aspect and a minimal mucoperiosteal flap is elevated exposing the crestal bone. With copious irrigation, sequential drilling prepares the appropriate width of the proposed implant. Care is taken after proper measurement from the x-ray not to perforate the antral floor. The depth of the drills can be measured on the panographic x-ray with suitable markers. After completion of the implant osteotomy site, the bone floor of the antrum is pushed superiorly to in-fracture the bone.

As explained below, an antral membrane expander is used to create and modify a cavity between the buccal bone and the medial wall of the sinus. The cavity is modified by the incorporation of a resorbable membrane therein. Graft material is inserted into the modified cavity and, after healing of the graft, an implant of appropriate length and width can be placed (e.g., within the prepared receptor site). Finally, in the case of insertion via the implant receptor site, the mucoperiosteal flap is repositioned and sutured.

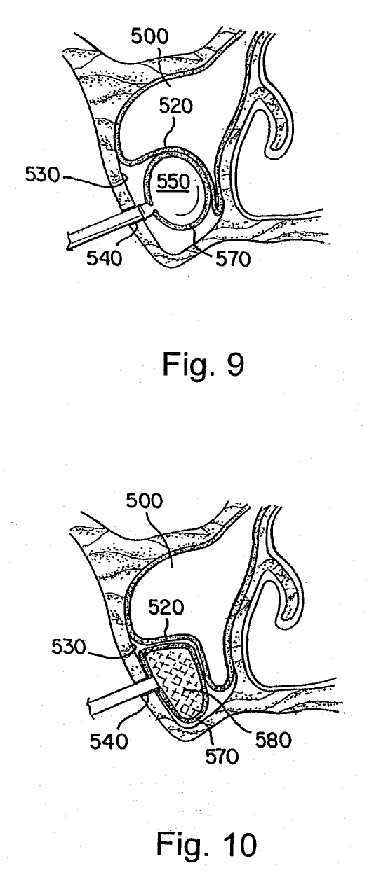

FIG. 5 shows a sectional illustration of the maxillary sinus 500 including the lateral maxillary wall 510, the sinus membrane 520, and the antral floor 530. Referring to FIG. 6, inserted through an aperture 540 formed in the maxillary wall 510, the deflated balloon 550 is initially positioned midway between the buccal bone and the medial wall of the sinus. Slow inflation of the balloon with saline lifts and elevates the membrane 520 from the antral floor 530 (FIG. 7). This results in an antral space 560 bordered superiorly by the reflected buccal bone and membrane, medially by the medial wall of the sinus, anterior/posterior by the non-reflected membrane and the roots of the adjacent teeth. The surgeon notes the amount of saline used to inflate the collagen antral membrane expander.

After the sinus membrane has been elevated to form the desired cavity, the balloon is reduced in size by pulling back on the syringe and withdrawn from the cavity 560 (FIG. 8). The deflated balloon is over-fitted with a collagen membrane 570. The collagen membrane encapsulated balloon is then inserted into the cavity and the balloon is re-inflated (FIG. 9). By inflating the membrane-encapsulated balloon, the collagen membrane 570 is pressed against the inner walls of the cavity. Again, the balloon is deflated and withdrawn from the cavity. However, the collagen membrane remains within the cavity to form a collagen membrane-modified cavity.

Finally, a syringe of appropriate graft volume, corresponding to the volume of the balloon used to elevate the membrane, is dispensed into the collagen membrane-modified cavity. The graft material 580 fills the cavity 560 and is substantially encapsulated by the collagen membrane 570 (FIG. 10). The syringe is detached and the incision is closed. After osseointegration of the graft material, implants (not shown) can be placed in the subantral space.

Advantageously, expansion of the membrane-encapsulated balloon presses the collagen membrane simultaneously against both the antral floor and the exterior surface of the sinus membrane. Thus, the collagen membrane-modified cavity comprises a collagen lining.

Due to the limited elasticity of the collagen membrane material, the collagen membrane capsule that is formed over the balloon can be folded or crimped. By folding or crimping the collagen membrane, the volume of the membrane capsule can expand during the expansion of the balloon without tearing or rupturing the collagen membrane.

When implanted into the human body, collagen membranes provide pathways for cellular movement and healing. Because the collagen membrane is porous, amino acids, mesenchymal cells, and small molecular weight compounds are free to traverse the membrane wall. Further, as a capsule formed over the balloon subantral membrane expander, cells can continue to grow on the collagen membrane to effectively patch tears and seal openings through the sinus membrane.

Advantageously, the collagen membrane is not permanent. Approximately one-quarter of all the protein in the human body is collagen. It is the main structural protein forming molecular cables that strengthen tendons, and resilient sheets of collagen support the skin and internal organs. Because it is a natural simple protein, the collagen membrane slowly breaks down into amino acids that are then absorbed by the body.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A resorbable collagen-based non-friable and conformable membrane comprising pores of a size suitable for free traversal of amino acids, mesenchymal cells and small molecular weight compounds across the membrane.

2. The resorbable collagen-based membrane of claim 1 wherein the pores are of insufficient size for bone graft material to freely traverse the membrane.

3. The resorbable collagen-based membrane of claim 1 wherein the membrane comprises type I collagen.

4. The resorbable collagen-based membrane of claim 1 comprising collagen fibers selected from the group consisting of bovine collagen fibers, porcine collagen fibers, equine collagen fibers, human collagen fibers and synthetic collagen fibers.

5. The resorbable collagen-based membrane of claim 4 wherein the bovine collagen fibers are isolated from the bovine corium.

6. The resorbable collagen-based membrane of claim 4, wherein the human collagen fibers are isolated from an Achilles tendon.

7. The resorbable collagen-based membrane of claim 1 wherein the membrane further comprises a bioactive agent.

8. The resorbable collagen-based membrane of claim 7 wherein the bioactive agent is a growth factor or platelet rich plasma.

9. The resorbable collagen-based membrane of claim 1 wherein the collagen-based membrane is programmed for resorption after at least 2-3 weeks of implantation into a living organism.

10. A resorbable capsule suitable for implantation into a cavity in a bone to be augmented, or a cavity between a bone to be augmented and an overlying membrane, wherein the capsule comprises a non-friable and conformable collagen-based membrane, the membrane comprising pores of a size suitable for free traversal of amino acids, mesenchymal cells and small molecular weight compounds across the membrane.

11. The resorbable capsule of claim 10 wherein the pores are of insufficient size for bone graft material to freely traverse the membrane.

12. The resorbable capsule of claim 10, wherein the collagen-based membrane comprises type I collagen.

13. The resorbable capsule of claim 10, wherein the collagen-based membrane comprises collagen fibers selected from the group consisting of bovine collagen fibers, porcine collagen fibers, equine collagen fibers, human collagen fibers or synthetic collagen fibers.

14. The resorbable capsule of claim 13 wherein the bovine collagen fibers are isolated from the bovine corium.

15. The resorbable capsule of claim 13, wherein the human collagen fibers are isolated from an Achilles tendon.

16. The resorbable capsule of claim 10 wherein the membrane further comprises a bioactive agent.

17. The resorbable capsule of claim 16 wherein the bioactive agent is a growth factor or platelet rich plasma.

18. The resorbable capsule of claim 10 wherein the collagen-based membrane is programmed for resorption after at least 2-3 weeks of implantation into the cavity.

19 The resorbable capsule of claim 10 wherein the cavity is between a buccal bone and a sinus membrane wall.

20. The resorbable capsule of claim 10 wherein the bone to be augmented is an alveolar bone.
